(19) United States
(12) Patent Application Publication
(10) Pub. No.: US 2006/0201212 A1
(43) Pub. Date: Sep. 14, 2006

(54) End cover assembly for a hollow structural member

(76) Inventors: Jeffery Van Zollinger, Kearneysville, WV (US); Meria H. Zollinger, Kearneysville, WV (US)

Correspondence Address: Ryan C. Stockett, 631 Tigers Eye Drive, Hedgesville, WV, 25427, US

(21) Appl. No.: 11/078,557

(22) Filed: Mar. 14, 2005

(51) Int. Cl. B65D 55/14; A61K 31/44; A01N 43/42

(52) U.S. Cl. 70/164

(57) ABSTRACT

An end cover assembly for a hollow structural member is disclosed. The end cover assembly has a cylindrical sleeve having at least one pair of through holes disposed in a radial surface of the cylindrical sleeve. The at least one pair of through holes are aligned with each other. The end cover assembly also has a plate member fixedly connected to an end of the cylindrical sleeve. The end cover assembly further has a securing device with at least one rod member and at least one locking mechanism. The at least one rod member is configured for insertion through the at least one pair of through holes. The locking mechanism is configured for removable connection to a first end of the at least one rod member to secure the end cover assembly to an end of the hollow structural member.

### TECHNICAL FIELD

The present disclosure relates generally to an end cover assembly, and more particularly, to an end cover assembly for a hollow structural member.

### BACKGROUND

Hollow structural members may be used for a variety of purposes including fluid transportation, material housing, or as a means for providing access. For example, a tube may be used as an air shaft in a mine to transport air into or out of the mine, or to provide access to the mine. A culvert may be used to direct fluid under a road. A well casing may house or provide access to a supply of fluid such as water, oil, or any other appropriate fluid. A pipe may provide access to underground utilities. In certain situations it may be important to secure an end of these hollow structural members to prevent unauthorized access, to minimize contamination, to curtail theft, or to limit tampering of the contents.

One method of securing a hollow structural member is described in U.S. Pat. No. 4,334,578 (the '578 patent) issued to Labrum on Jun. 15, 1982. The '578 patent describes a closure for a well casing. The closure includes a hollow cylindrical member having a cover on one end and being open on the other end to fit over the top of the well casing. The closure includes a plurality of downwardly extending locking members, which are welded to a flat top member of the cover. A segment of casing pipe is welded to the top of the well casing. Welded to an inside diameter of the casing segment are a plurality of angularly disposed locking lugs, which cooperate with the downwardly extending locking members in order to lock the closure onto the casing segment. A locking mechanism is included to prevent the locking members from disengaging from the locking lugs.

Although the closure of the '578 patent may minimize unauthorized access to a well casing, it may be complex, expensive, and difficult to retrofit to an existing hollow structural member. In particular, the closure of the '578 patent includes many different components and may require multiple manufacturing processes to assemble. The many different components and multiple manufacturing processes increase the complexity and cost of the closure. Further, because the closure of the '578 patent requires a welding process, the closure may be difficult to add to an existing well casing or to a well casing that is non-metallic.

The disclosed tube end cover assembly is directed to overcoming one or more of the problems set forth above.

### SUMMARY OF THE INVENTION

In one aspect, the present disclosure is directed to an end cover assembly for a hollow structural member. The end cover assembly includes a cylindrical sleeve having at least one pair of through holes disposed in a radial surface of the cylindrical sleeve. The at least one pair of through holes are aligned with each other. The end cover assembly also includes a plate member fixedly connected to an end of the cylindrical sleeve, and a securing device having at least one rod member and at least one locking mechanism. The at least one rod member is configured for insertion through the at least one pair of through holes. The locking mechanism is configured for removable connection to a first end of the at least one rod member to secure the end cover assembly to an end of the hollow structural member.

In another aspect, the present disclosure is directed to a securing device that includes a rod member at least 5 inches long. The rod member has a flange element on one end. The securing device also includes a locking mechanism configured to removably connect to a second end of the rod member.

In yet another aspect, the present disclosure is directed to a securing device that includes at least one rod member, a first locking mechanism, and a second locking mechanism. At least one of the first and second locking mechanisms is removably connectable to the at least one rod member.

In yet another aspect, the present disclosure is directed to an end cap. The end cap includes a cylindrical sleeve having at least on pair of through holes disposed in a radial surface of the cylindrical sleeve. The at least one pair of through holes are aligned with each other. The end cap also includes a plate member fixedly connected to an end of the cylindrical sleeve.

In yet another aspect, the present disclosure is directed to a method of securing an end of a hollow structural member. The method includes placing a cylindrical sleeve over an end of the hollow structural member. The cylindrical sleeve has at least one pair of through holes. The method also includes making a pair of holes through a wall of the hollow structural member. The holes are located to align with the at least one pair of holes in the cylindrical sleeve. The method also includes inserting a rod member through the at least one pair of holes in the cylindrical sleeve and the pair of holes in the hollow structural member. The method further includes securing a locking mechanism to an end of the rod member.

### BRIEF DESCRIPTION OF THE DRAWINGS

FIG. 1 is a pictorial illustration of an exemplary disclosed end cover assembly;

FIG. 1A is a side cross-sectional view illustration of the end cover assembly of FIG. 1;

FIG. 1B is a top cross-sectional view illustration of the end cover assembly of FIG. 1;

FIG. 2 is a pictorial illustration of another exemplary disclosed end cover assembly;

FIG. 2A is a side cross-sectional view illustration of the end cover assembly of FIG. 2;

FIG. 3 is a pictorial illustration of an exemplary disclosed securing device for the end cover assembly of FIGS. 1 and 2;

FIG. 4 is a pictorial illustration of another exemplary disclosed securing device for the end cover assembly of FIGS. 1 and 2;

FIG. 5 is a pictorial illustration of another exemplary disclosed securing device for the end cover assembly of FIGS. 1 and 2; and

FIG. 6 is a pictorial illustration of another exemplary disclosed securing device for the end cover assembly of FIGS. 1 and 2.

### DETAILED DESCRIPTION

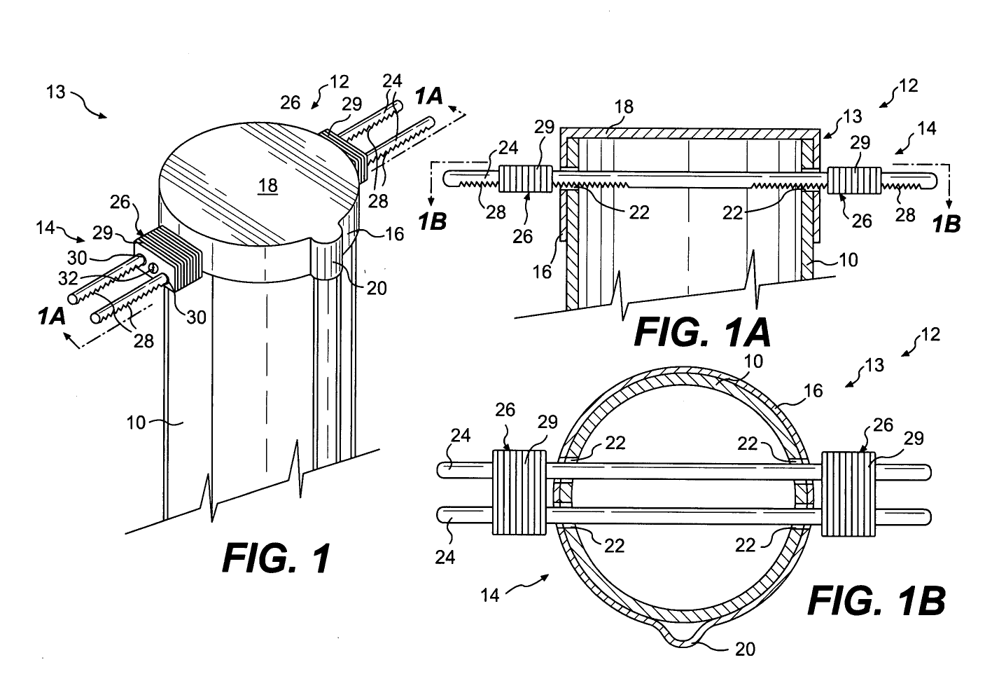

FIG. 1 illustrates a hollow structural member 10 having an exemplary end cover assembly 12. Hollow structural member 10 may include any hollow structural member that is used for transportation or housing of materials, for providing access, or for any other suitable purpose. For example, hollow structural member 10 may include a well casing for housing a fluid such as water, oil, or any type of fluid. Hollow structural member 10 may also embody a culvert, a mine shaft, an air vent, a utility tube, or any other type of hollow structural member known in the art.

End cover assembly 12 may include multiple components that cooperate to secure an end of hollow structural member 10. Specifically, end cover assembly 12 may include an end cap 13 and a securing device 14. End cover assembly 12 may be removably connected to the end of hollow structural member 10 to prevent unauthorized access, to minimize contamination, to curtail theft, or to limit tampering of the contents.

End cap 13 may include a cylindrical sleeve 16, and an end plate 18. Cylindrical sleeve 16 and/or end plate 18 may be a single integral component fabricated, for example, through a casting process, a thermal molding process, an injection molding process, or any other fabrication process known in the art. It is contemplated that cylindrical sleeve 16 and/or end plate 18 may alternatively initially embody separate components that are joined together by way of a welding process, a brazing process, an adhesive, threaded fastening, or in any other appropriate manner.

Cylindrical sleeve 16 may be weather resistant and configured to receive an end of hollow structural member 10. In particular, cylindrical sleeve 16 may be manufactured from a weather resistant material such as, for example aluminum, stainless steel, resin, or any other material that is suitably weather resistant. Cylindrical sleeve 16 may have an inner diameter that is larger than an outer diameter of hollow structural member 10. In one example, cylindrical sleeve 16 has an inner diameter of at least 5 inches, and more preferably at least 7 inches to accommodate residential type well casings. In one example, cylindrical sleeve 16 may include a projection 20 configured to accommodate an electrical connection for a pump (not shown) of the type generally disposed toward the bottom of a well casing

As illustrated in FIGS. 1A and 1B, cylindrical sleeve 16 may be configured to receive securing device 14. Specifically, cylindrical sleeve 16 may include one or more pairs of through holes 22. The holes 22 of each pair may be axially aligned with each other to facilitate the operation of securing device 14. Although FIG. 1A illustrates the two pairs of through holes 22 as being horizontally aligned, the two pairs of through holes 22 may alternatively be vertically aligned, or horizontally and vertically misaligned. It is contemplated that cylindrical sleeve 16 may alternatively include a greater or lesser number of pairs of through holes 22 than what is depicted if FIGS. 1A and 1B. While the diameters of through holes 22 may be of any size sufficient to accommodate securing device 14, a slip-fit clearance may minimize the likelihood of access, contamination, or tampering. It is contemplated that grommets (not shown) may be disposed within through holes 22 and around securing device 14 to further minimize the likelihood of access, contamination, or tampering. It is also contemplated that a sealing device such as, for example, an o-ring (not shown) may be disposed between an inner surface of cylindrical sleeve 16 and an outer surface of hollow structural member 10 to further secure the end of hollow structural member 10.

End plate 18 may be manufactured from a weather resistant material and configured to engage an end of hollow structural member 10. Similar to cylindrical sleeve 16, end plate 18 may be fabricated from aluminum, stainless steel, resin, or any other material that is suitably weather resistant. End plate 18 may engage the end of hollow structural member 10 as end cover assembly 12 is slidingly disposed over the end of hollow structural member 10. It is contemplated that a sealing device such as a gasket (not shown) or an o-ring (not shown) may be disposed between end plate 18 and the end of hollow structural member 10 to further secure the end of hollow structural member 10.

Securing device 14 may be configured for insertion through holes 22 to secure end cover assembly 12 to hollow structural member 10. As illustrated in the examples of FIGS. 1-1B, securing device 14 may include two rod members 24 and two locking mechanisms 26. Each of rod members 24 may be inserted through a pair of though holes 22 and one locking mechanism 26 joined to an end of each of rod members 24 to lock end cover assembly 12 in place.

Rod member 24 may include an elongated cylindrical rod having recesses 28 such as grooves or notches disposed on each end of rod member 24. Rod member 24 may be fabricated from a weather resistant hardened material or alternatively may be coated with a weather resistant coating. Each rod member 24 may extend at least the diameter of cylindrical sleeve 16. It is contemplated that instead of recesses 28, rod members 24 may alternatively include protrusions on each end of rod member 24.

Each of locking mechanisms 26 may be substantially identical and configured to engage and release recesses 28 of rod members 24. Specifically, locking mechanism 26 may embody lock body 29 having two receiving portions 30 and a releasing mechanism 32 (referring to FIG. 1). One end of each rod member 24 may be inserted through a receiving portion 30 of locking mechanism 26 to engage recesses 28 of rod member 24 with a protrusion (not shown) of lock body 29. As rod member 24 is inserted through receiving portion 30, the protrusions of lock body 29 may automatically secure rod member 24 to lock body 29. Without actuating releasing mechanism 32, rod member 24 may only be inserted further into lock body 29. Releasing mechanism 32 may include a tumbler (not shown) that is configured to receive a key (not shown). The key may be turned to release rod member 24 by disengaging the protrusion from recess 28. It is contemplated that releasing mechanism 32 may alternatively embody a combination button-type of releasing mechanism wherein a combination of buttons may be pushed to release rod member 24, a combination wheel-type of releasing mechanism wherein a pattern of wheel positions releases rod member 24, or any other type of releasing mechanism known in the art. It is further contemplated that locking mechanism 26 may be fabricated from a hardened weather resistant material, may be coated with a weather resistant material, and/or that a weather resistant jacket may be disposed about an external surface of locking mechanism 26.

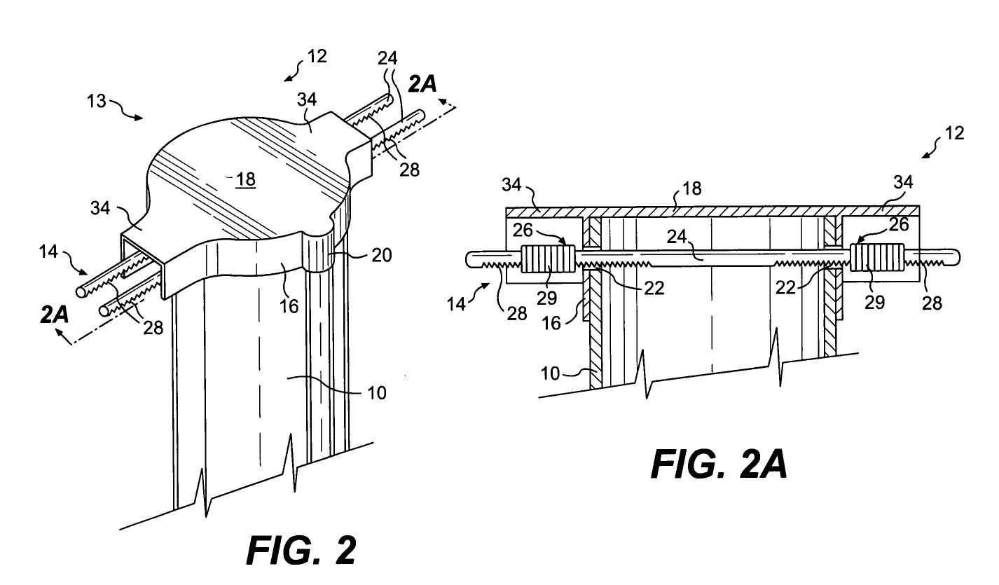

FIG. 2 illustrates an alternate embodiment for end cap 13, wherein securing locking mechanisms 26 may be at least somewhat shielded from weather. In this embodiment, end cap 13 may include two three-sided protrusions 34 disposed on opposing portions of cylindrical sleeve 16 to protect each locking mechanism 26 when end cover assembly 12 is secured to the end of hollow structural member 10. In particular, each protrusion 34 may be configured to shield an end of securing device 14 from an upward axial direction relative to hollow structural member 10, and from two opposing transverse side directions. Protrusions 34 may be integral to cylindrical sleeve 16 and end cap 13 or, alternatively, may be separate and joined through a welding process, a brazing process, an adhesive process, or any other suitable fabrication process. It is contemplated that one of protrusions 34 may be omitted if desired, such that only one end of securing device 14 may be protected from weather. It is also contemplated that protrusions 34 may alternatively embody a four-sided structure, wherein locking mechanism 26 is further protected from an axially downward direction relative to hollow structural member 10.

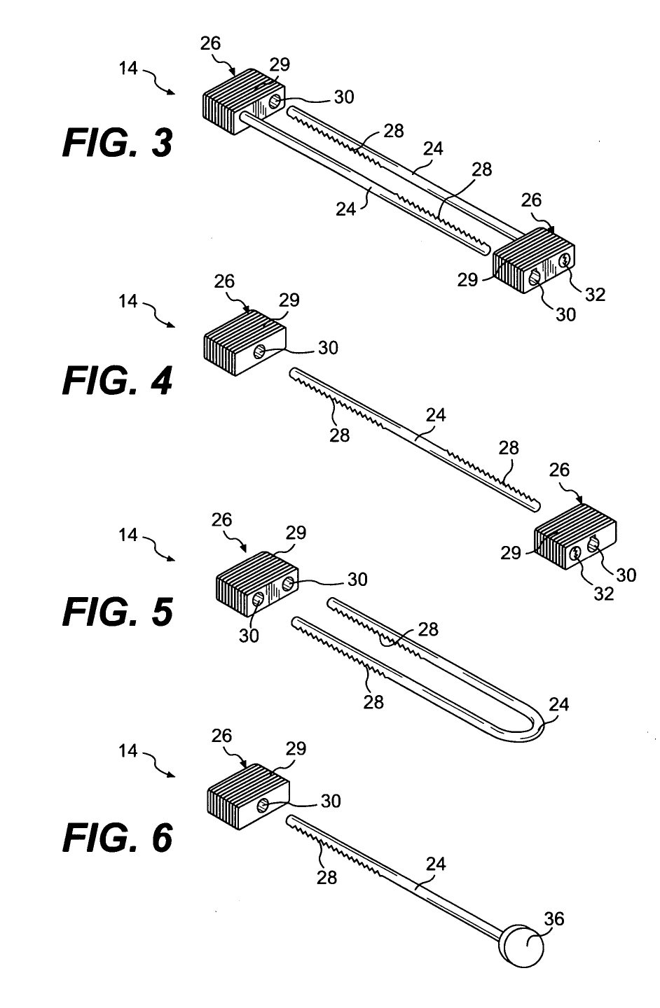

FIG. 3 illustrates an alternate embodiment for securing device 14. Similar to securing device 14 of FIGS. 1-2B, securing device 14 of FIG. 3 includes two rod members 24 and two locking mechanisms 26. However, in contrast to the embodiment of FIGS. 1-2B, FIG. 3 illustrates one rod member 24 being fixedly connected to lock body 29 of each locking mechanism 26. In this embodiment, the remaining free end of each rod member 24 is insertable through a pair of through holes 22 of cylindrical sleeve 16 and a pair of through holes of hollow structural member 10 to removably connect to the other lock body 29.

FIG. 4 illustrates another alternate embodiment for securing device 14. Similar to securing device 14 of FIGS. 1-2B, securing device 14 of FIG. 3 includes two locking mechanisms 26. However, in contrast to the embodiment of FIGS. 1-2B, FIG. 4 illustrates only a single rod member 24, each end of the single rod member 24 engaging one of the two locking mechanisms 26. In this embodiment, each locking mechanism 26 only includes a single receiving portion 30. Receiving portion 30 may or may not be centrally located within lock body 29. It is contemplated that each locking mechanism 26 may still include two receiving portions 30, only one of which is utilized in the embodiment of FIG. 4. In the situation where locking mechanism 26 still includes two receiving portions 30, one of the two receiving portions may or may not be plugged to minimize internal exposure of lock body 29 to weather.

FIG. 5 illustrates another alternate embodiment for securing device 14. Similar to securing device 14 of FIG. 4, securing device 14 of FIG. 5 includes a single rod member 24. However, in contrast to the embodiment of FIG. 4, securing device 14 of FIG. 5 only includes a single locking mechanism 26. In this embodiment, the single rod member 24 may be formed in a U-shape such that recesses 28 located at each end of rod member 24 may be configured to engage the single locking mechanism 26. In order to extend completely through cylindrical sleeve 16, with the curved end of the U-shaped rod member 24 engaging one portion of cylindrical sleeve 16 and the free ends of rod member 24 protruding from an opposing portion of cylindrical sleeve 16 for engagement with locking mechanism 26, each leg of the U-shaped rod member 24 must be longer than the diameter of cylindrical sleeve 16.

FIG. 6 illustrates another alternate embodiment for securing device 14. Similar to securing device 14 of FIG. 5, securing device 14 of FIG. 6 includes a single rod member 24 and a single locking mechanism 26. However, in contrast to the embodiment of FIG. 5, rod member 24 of FIG. 6 is straight and includes recesses 28 on only one end. In further contrast to FIG. 5, rod member 24 of FIG. 6 also includes a flange element 36 fixedly connected to the end of rod member 24 without recesses 28. Flange element 36 may have a shape configured to prevent insertion through holes 22. For example, flange element 36 may be generally circular, with a diameter greater than a diameter of holes 22. It is contemplated that flange element 36 may alternatively be any other appropriate shape that prevents insertion through holes 22 such as, for example, square, triangular, T-shaped, or any other appropriate shape.

### INDUSTRIAL APPLICABILITY

The disclosed end cover assembly may be applicable to any hollow structural member where minimizing access, contamination, theft, and tampering may be desirable. The disclosed end cover assembly may be a simple cost effective means for securing an end of the hollow structural member. In addition, the disclosed end cover assembly may be easily retrofitted to most hollow structural members with few required modifications to the hollow structural member. The connection of end cover assembly 12 to hollow structural member 10 will now be described.

End cap 13 may be placed over an end of hollow structural member 10 in preparation for making modifications to hollow structural member 10. For example, cylindrical sleeve 16 may be axially aligned with hollow structural member 10 and slid downward into place on hollow structural member 10 until end plate 18 comes into contact with the end of hollow structural member 10. Markings may then be made on hollow structural member 10 through holes 22, in preparation for making holes within hollow structural member 10 that align with holes 22. End cap 13 may then either be removed prior to making the holes within hollow structural member 10 at the location previously marked or, alternatively, end cap 13 may remain in place and the holes within hollow structural member 10 made through holes 22 with holes 22 acting as guides during the hole making process. The holes within hollow structural member 10 may be made by way of drilling, boring, punching, cutting, torching, or in any other appropriate manner known in the art.

Once the holes within hollow structural member 10 have been made, end cap 13 may be repositioned over the end of hollow structural member 10 if end cap 13 was previously removed. Rod member(s) 24 may then be inserted through holes 22 and through the newly made holes of hollow structural member 10. Locking mechanism(s) 26 may then be pushed onto the free ends of rod member(s) 24 to allow the protrusion within lock body 29 to engage recess 28, thereby securing end cap 13 to hollow structural member 10.

During the making of holes within hollow structural member 10, it may be desirable to minimize contamination of hollow structural member 10 with debris generated from the hole-making process. In one example, a means for blocking debris may be inserted into hollow structural member 10 to a point within hollow structural member 10 past the intended hole location relative to the end of hollow structural member 10. The means for blocking debris may include, among other things, a bag, a cupping member, a foam plug, or any other suitable device configured to catch the debris generated from the hole making process, while remaining in position within hollow structural member 10.

It will be apparent to those skilled in the art that various modifications and variations can be made to the disclosed end cover assembly. Other embodiments will be apparent to those skilled in the art from consideration of the specification and practice of the disclosed end cover assembly. It is intended that the specification and examples be considered as exemplary only, with a true scope being indicated by the following claims and their equivalents.

What is claimed is:

1. An end cover assembly for a hollow structural member, comprising:
a cylindrical sleeve having at least one pair of through holes disposed in a radial surface of the cylindrical sleeve, the at least one pair of through holes being aligned with each other;
a plate member fixedly connected to an end of the cylindrical sleeve; and
a securing device having at least one rod member and at least one locking mechanism, wherein the at least one rod member is configured for insertion through the at least one pair of through holes and the locking mechanism is configured for removable connection to a first end of the at least one rod member to secure the end cover assembly to an end of the hollow structural member

2. The end cover assembly of claim 1, wherein the cylindrical sleeve has an inner diameter greater than an outer diameter of the hollow structural member and the cylindrical sleeve is configured to internally receive the end of the hollow structural member.

3. The end cover assembly of claim 2, wherein the hollow structural member includes at least one pair of through holes, the at least one pair of through holes of the cylindrical sleeve are with the at least one pair of through holes of the hollow structural member when the cylindrical sleeve is received within the hollow structural member, and the at least one rod member is inserted through at least one pair of holes in the hollow structural member when the end cover assembly is connected to the hollow structural member.

4. The end cover assembly of claim 1, wherein the at least one rod member includes at least one of protrusions or recesses configured to engage the locking mechanism.

5. The end cover assembly of claim 1, wherein the at least one pair of through holes is a first pair of through holes, the cylindrical sleeve includes a second pair of through holes disposed in the radial surface of the cylindrical sleeve, the at least one rod member is a first rod member, the at least one locking mechanism is a first locking mechanism, and the securing device further includes:
a second rod member configured for insertion through the second pair of through holes; and
a second locking mechanism.

6. The end cover assembly of claim 5, wherein the first rod member includes a first end fixedly connected to the second locking mechanism, the second rod member includes a first end fixedly connected to the first locking mechanism and a second end removably connectable to the second locking mechanism.

7. The end cover assembly of claim 5, wherein each of the first and second rod members are removably connectable to each of the first and second locking mechanisms.

8. The end cover assembly of claim 1, wherein the at least one rod member is substantially U-shaped and includes a second end removably connectable to the at least one locking mechanism.

9. The end cover assembly of claim 1, wherein the at least one locking mechanism is a first locking mechanism and the end cover assembly further includes a second locking mechanism removably connectable to a second end of the at least one rod member.

10. The end cover assembly of claim 1, wherein the at least one rod member includes a second end having a flange element blocking the second end from insertion through the at least one pair of holes.

11. The end cover assembly of claim 1, wherein the at least one rod member has a length greater than the diameter of the hollow structural member.

12. The end cover assembly of claim 11, wherein the diameter of the hollow structural member is at least 5 inches.

13. The end cover assembly of claim 1, further including at least one protrusion disposed proximal at least one of the at least one pair of through holes, the at least one protrusion configured to at least partially enclose the locking mechanism when the end cover assembly is secured to the hollow structural member.

14. The end cover assembly of claim 13, wherein the at least one protrusion forms at least a three sided structure.

15. A securing device comprising:
a rod member at least 5 inches long and having a flange element on one end;
a locking mechanism configured to removably connect to a second end of the rod member.

16. A securing device comprising:
at least one rod member
a first locking mechanism; and
a second locking mechanism, wherein at least one of the first and second locking mechanisms is removably connectable to the at least one rod member.

17. The securing device of claim 16, wherein each of the first and second locking mechanisms are removably connectable to the at least one rod member.

18. The securing device of claim 16, wherein the at least one rod member is a first rod member, the first locking mechanism is removably connected to the first rod member, the second locking mechanism is fixedly connected to the first rod member, the securing device further includes a second rod member, the first locking mechanism is fixedly connected to the second rod member, and the second locking mechanism is removably connected to the second rod member.

19. The securing device of claim 16, wherein the at least one rod member is a first rod member, the securing device further includes a second rod member, and the first and second locking mechanisms are each removably connected to both of the first and second rod members.

20. An end cap, comprising:
a cylindrical sleeve having at least on pair of through holes disposed in a radial surface of the cylindrical sleeve, the at least one pair of through holes being aligned with each other; and
a plate member fixedly connected to an end of the cylindrical sleeve.

21. The end cap of claim 20, further including at least one protrusion proximally disposed relative to one of the at least one pair of through holes.

22. The end cap of claim 21, wherein the at least one protrusion is a first protrusion and the end cap includes a second protrusion proximally disposed relative to the other of the at least one pair of through holes.

23. The end cap of claim 21, wherein the at least one protrusion includes a three-sided structure.

24. A method of securing an end of a hollow structural member, the method comprising:
placing a cylindrical sleeve over an end of the hollow structural member, the cylindrical sleeve having at least one pair of through holes;
making a pair of holes through a wall of the hollow structural member, the holes located to align with the at least one pair of holes in the cylindrical sleeve;
inserting a rod member through the at least one pair of holes in the cylindrical sleeve and the pair of holes in the hollow structural member; and
securing a locking mechanism to an end of the rod member.

25. The method of claim 24, further including securing a second locking mechanism to a second end of the rod member.

26. The method of claim 24, wherein the at least one pair of through holes in the cylindrical sleeve is a first pair of through holes, the cylindrical sleeve has a second pair of through holes, and the method further includes making a second pair of holes through the wall of the hollow structural member, the second pair of through holes located to align with the second pair of through holes in the cylindrical sleeve.

27. The method of claim 26, further including:
inserting a second rod member through the second pair of through holes in the cylindrical sleeve and the second pair of holes in the hollow structural member; and
securing a second locking mechanism to an end of the second rod member.

28. The method of claim 27, further including:
securing the locking mechanism to an end of the second rod member; and
securing the second locking mechanism to an end of the rod member.

29. The method of claim 24, further including collecting the debris generated from the making of the through holes to minimize contamination of the hollow structural member.
